(19) United States
(12) Patent Application Publication
(10) Pub. No.: US 2026/0174506 A1
(43) Pub. Date: Jun. 25, 2026

(54) AUGMENTED REALITY NAVIGATION BASED ON MEDICAL IMPLANTS

(71) Applicant: Augmedics Ltd., Yokneam Illit (IL)

(72) Inventors: Stuart WOLF, Yokneam (IL); Tomer GERA, Kfar Tavor (IL); Boris NEMIROVSKY, Ramat Ishay (IL)

(21) Appl. No.: 19/540,459

(22) Filed: Feb. 13, 2026

Related U.S. Application Data
(63) Continuation of application No. PCT/IB2024/057929, filed on Aug. 15, 2024.
(60) Provisional application No. 63/520,215, filed on Aug. 17, 2023.

(51) Int. Cl. A61B 34/20; A61B 34/10; A61B 90/00

(52) U.S. Cl. CPC A61B 34/20; A61B 34/10; A61B 90/37; A61B 2090/365

(57) ABSTRACT

The disclosure generally relates to systems, devices, and methods to facilitate image-guided medical treatment and/or diagnostic procedures (e.g., surgery or other interventions among other considered medical usages), including using augmented reality overlay images that include one or more guidance attributes for guiding the implantation of a medical device.

### CROSS-REFERENCE TO RELATED APPLICATIONS

This application is a continuation of PCT International Application PCT/IB2024/057929, filed Aug. 15, 2024, titled “Augmented Reality Navigation Based On Medical Implants,” which claims priority to U.S. Provisional Application No. 63/520,215, filed Aug. 17, 2023, titled “Augmented Reality Navigation Based On Medical Implants,” the entire contents of each of which are hereby incorporated by reference.

### FIELD

The disclosure generally relates to systems, devices, and methods to facilitate image-guided medical treatments and/or diagnostic procedures (e.g., surgery or other interventions among other considered medical or diagnostic usages), including for implantation of medical devices.

### BACKGROUND

Image-guided surgery employs tracked surgical tools or instruments and images of the patient anatomy in order to guide the procedure. In such procedures, a proper and current imaging or visualization of regions of interest of the patient anatomy is of high importance.

Near-eye display devices and systems, such as head-mounted displays including special-purpose eyewear (e.g., glasses), are used in augmented reality systems.

See-through displays (e.g., displays including at least a portion that is see-through) are used in augmented reality systems, for example for performing image-guided and/or computer-assisted surgery. Typically, but not necessarily, such see-through displays are near-eye displays (e.g., integrated in a Head-Mounted Device (HMD)). In this way, a computer-generated image may be presented to a healthcare professional who is performing the procedure, such that the image is aligned with an anatomical portion of a patient who is undergoing the procedure. Systems of this sort for image-guided surgery are described, for example, in U.S. Pat. Nos. 9,928,629, 10,835,296, 10,939,977, PCT International Publication WO 2019/211741, U.S. Patent Application Publication 2020/0163723, and PCT International Publication WO 2022/053923. The disclosures of all these patents and publications are incorporated herein by reference.

### SUMMARY

Disclosed herein are various embodiments of augmented reality surgical systems that can be used to, among other things, assist a healthcare provider in implantation of medical devices, such as, for example, in guiding of a rod between pedicle screws in spinal fusion surgery. The systems, methods, and devices disclosed herein can provide for a more efficient, more accurate, and/or more safe surgical procedure.

According to some embodiments, an augmented reality surgical display system for guiding implantation of a medical device includes: a see-through display configured to overlay augmented reality images onto reality; one or more cameras; and at least one processor configured to: detect and store in a memory locations of a plurality of medical device fixation locations in a patient; calibrate a medical device distal tip location with respect to a handle marker; track, using the one or more cameras during a surgical procedure, a location of the handle marker; determine a location of the medical device distal tip based on the tracked location of the handle marker; generate one or more guidance attributes (e.g., navigational guides) based on the determined location of the medical device distal tip and the stored locations of one or more of the plurality of medical device fixation locations; and display on the see-through display, aligned with reality, the following: an indication of the determined location of the medical device distal tip; an indication of locations of one or more of the plurality of medical device fixation locations; and the one or more guidance attributes. In some implementations, another portion of the medical device may be used other than the distal tip.

In some embodiments, the at least one processor is further configured to: generate a 3D virtual model of a body of the medical device; and further display on the see-through display, aligned with reality, at least a portion of the generated 3D virtual model of the body of the medical device.

In some embodiments, the system further includes a depth sensor (e.g., one or a plurality of depth sensors or a depth sensing or depth mapping system), and wherein the at least one processor is configured to generate the 3D virtual model of the body of the medical device using data output from the depth sensor.

In some embodiments, the at least one processor is configured to generate the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images or other medical images.

In some embodiments, the generation of the one or more guidance attributes includes comparing the determined location of the medical device distal tip (or other portion of the medical device) to a location of one of the plurality of medical device fixation locations, to determine a direction in which the medical device distal tip (or other portion of the medical device) should be moved to reach the one of the plurality of medical device fixation locations.

In some embodiments, the generation of the one or more guidance attributes includes comparing the determined location of the medical device distal tip (or other portion of the medical device) to a location of one of the plurality of medical device fixation locations, to determine a distance by which the medical device distal tip (or other portion of the medical device) should be moved to reach the one of the plurality of medical device fixation locations.

In some embodiments, the one or more guidance attributes are not generated based on a deviation from a preoperative planned trajectory but are determined in real time.

In some embodiments, each of the plurality of medical device fixation locations includes a fixation location of a pedicle screw, and wherein the medical device comprises a rod shaped to be affixed to the plurality of medical device fixation locations.

In some embodiments, the one or more guidance attributes (e.g., navigational guides) include one or more directional indicators indicative of one or more of the following: a direction in which the medical device distal tip should be moved, or a direction in which a proximal end of the medical device should be moved. Another portion of the medical device may be used for the guidance attribute or navigational guide as well.

In some embodiments, at least one of the one or more directional indicators includes an arrow, although other indicators or graphical icons may be used.

In some embodiments, the one or more guidance attributes (e.g., navigational guides) comprise a distance of the medical device distal tip to one of the plurality of medical device fixation locations.

According to some embodiments, an augmented reality surgical display system for guiding implantation of a medical device includes: a see-through display configured to overlay augmented reality images onto reality; one or more cameras; a depth sensor; and at least one processor configured to: detect and store in a memory locations of a plurality of medical device fixation locations in a patient; track, using the depth sensor during a surgical procedure, a location of a distal tip of the medical device; generate one or more guidance attributes (e.g., navigational guides) based on the tracked location of the distal tip of the medical device and the stored locations of one or more of the plurality of medical device fixation locations; and display on the see-through display, aligned with reality, the following: an indication of the tracked location of the distal tip of the medical device; an indication of locations of one or more of the plurality of medical device fixation locations; and the one or more guidance attributes.

In some embodiments, the at least one processor is further configured to: generate a 3D virtual model of a body of the medical device; and further display on the see-through display, aligned with reality, at least a portion of the generated 3D virtual model of the body of the medical device.

In some embodiments, the at least one processor is configured to generate the 3D virtual model of the body of the medical device using data output from the depth sensor.

In some embodiments, the at least one processor is configured to generate the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images.

In some embodiments, the generation of the one or more guidance attributes includes comparing the tracked location of the distal tip of the medical device to a location of one of the plurality of medical device fixation locations, to determine a direction in which the distal tip of the medical device should be moved to reach the one of the plurality of medical device fixation locations.

In some embodiments, the generation of the one or more guidance attributes includes comparing the tracked location of the distal tip of the medical device to a location of one of the plurality of medical device fixation locations, to determine a distance by which the distal tip of the medical device should be moved to reach the one of the plurality of medical device fixation locations.

In some embodiments, the one or more guidance attributes are not generated based on a deviation from a preoperative planned trajectory.

In some embodiments, each of the plurality of medical device fixation locations includes a fixation location of a pedicle screw, and wherein the medical device comprises a rod shaped to be affixed to the plurality of medical device fixation locations.

In some embodiments, the one or more guidance attributes (e.g., navigational guides) include one or more directional indicators indicative of one or more of the following: a direction in which the distal tip of the medical device should be moved, or a direction in which a proximal end of the medical device should be moved.

In some embodiments, at least one of the one or more directional indicators is an arrow.

In some embodiments, the one or more guidance attributes (e.g., navigational guides) include a distance of the distal tip of the medical device to one of the plurality of medical device fixation locations.

In some embodiments, the at least one processor is configured to detect the locations of the plurality of medical device fixation locations using data output from the depth sensor.

According to some embodiments, a method of guiding implantation of a medical device includes detecting and storing, in a memory, locations of a plurality of medical device fixation locations (optionally, in a patient); detecting a relationship between a medical device distal tip and a handle marker, to calibrate the medical device distal tip with respect to the handle marker; tracking, using one or more cameras (optionally, during a surgical procedure), a location of the handle marker; determining a location of the medical device distal tip based on the tracked location of the handle marker; generating, by at least one processor, one or more guidance attributes (e.g., navigational guides) based on the determined location of the medical device distal tip and the stored locations of one or more of the plurality of medical device fixation locations; and displaying on a see-through display, aligned with reality, the following: an indication of the determined location of the medical device distal tip; an indication of locations of one or more of the plurality of medical device fixation locations; and the one or more guidance attributes.

In some embodiments, the method further includes generating a 3D virtual model of a body of the medical device; and further displaying on the see-through display, aligned with reality, at least a portion of the generated 3D virtual model of the body of the medical device.

In some embodiments, the method further includes generating the 3D virtual model of the body of the medical device using data output from a depth sensor.

In some embodiments, the method further includes generating the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images.

In some embodiments, generating the one or more guidance attributes includes comparing the determined location of the medical device distal tip to a location of one of the plurality of medical device fixation locations, to determine a direction in which the medical device distal tip should be moved to reach the one of the plurality of medical device fixation locations.

In some embodiments, generating the one or more guidance attributes includes comparing the determined location of the medical device distal tip to a location of one of the plurality of medical device fixation locations, to determine a distance by which the medical device distal tip should be moved to reach the one of the plurality of medical device fixation locations.

In some embodiments, the one or more guidance attributes are not generated based on a deviation from a preoperative planned trajectory.

In some embodiments, each of the plurality of medical device fixation locations includes a fixation location of a pedicle screw, and wherein the medical device comprises a rod shaped to be affixed to the plurality of medical device fixation locations.

In some embodiments, the one or more guidance attributes (e.g., navigational guides) include one or more directional indicators indicative of one or more of the following: a direction in which the medical device distal tip should be moved, or a direction in which a proximal end of the medical device should be moved.

In some embodiments, at least one of the one or more directional indicators is an arrow or other graphical indicator or icon.

In some embodiments, the one or more guidance attributes (e.g., navigational guides) include a distance of the medical device distal tip to one of the plurality of medical device fixation locations.

According to some embodiments, a method of guiding implantation of a medical device includes: detecting and storing, in a memory, locations of a plurality of medical device fixation locations (optionally, in a patient); tracking, using a depth sensor (optionally, during a surgical procedure), a location of a distal tip of the medical device; generating, by at least one processor, one or more guidance attributes (e.g., navigational guides) based on the tracked location of the distal tip of the medical device and the stored locations of one or more of the plurality of medical device fixation locations; displaying on a see-through display, aligned with reality, the following: an indication of the tracked location of the distal tip of the medical device; an indication of locations of one or more of the plurality of medical device fixation locations; and the one or more guidance attributes.

In some embodiments, the method further includes generating a 3D virtual model of a body of the medical device; and further displaying on the see-through display, aligned with reality, at least a portion of the generated 3D virtual model of the body of the medical device.

In some embodiments, the method further includes generating the 3D virtual model of the body of the medical device using data output from the depth sensor.

In some embodiments, the method further includes generating the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images.

In some embodiments, generating the one or more guidance attributes includes comparing the tracked location of the distal tip of the medical device to a location of one of the plurality of medical device fixation locations, to determine a direction in which the distal tip of the medical device should be moved to reach the one of the plurality of medical device fixation locations.

In some embodiments, generating the one or more guidance attributes includes comparing the tracked location of the distal tip of the medical device to a location of one of the plurality of medical device fixation locations, to determine a distance by which the distal tip of the medical device should be moved to reach the one of the plurality of medical device fixation locations.

In some embodiments, the one or more guidance attributes are not generated based on a deviation from a preoperative planned trajectory.

In some embodiments, each of the plurality of medical device fixation locations includes a fixation location of a pedicle screw, and wherein the medical device comprises a rod shaped to be affixed to the plurality of medical device fixation locations.

In some embodiments, the one or more guidance attributes (e.g., navigational guides) include one or more directional indicators indicative of one or more of the following: a direction in which the distal tip of the medical device should be moved, or a direction in which a proximal end of the medical device should be moved.

In some embodiments, at least one of the one or more directional indicators is an arrow.

In some embodiments, the one or more guidance attributes (e.g., navigational guides) include a distance of the distal tip of the medical device to one of the plurality of medical device fixation locations.

In some embodiments, the detecting the locations of the plurality of medical device fixation locations includes using data output from the depth sensor.

According to some embodiments, a system for generating a shape of an implantable medical device includes: a tracking system capable of detecting locations of a plurality of markers that are each in a fixed relationship with respect to a medical device fixation location of a plurality of medical device fixation locations in a patient; and one or more processors configured to: detect a location of each of the plurality of markers using the tracking system; determine a location of each of the medical device fixation locations based on the detected marker locations and the fixed relationships; generate a set of points in three-dimensional space that together define a curved line that passes through all of the determined locations of the medical device fixation locations; and output the generated set of points for use in shaping the implantable medical device.

In some embodiments, the set of points is generated during a surgical procedure that affixes the medical device fixation locations to the patient, and the set of points is generated based on the determined locations determined during the surgical procedure, not during preoperative planning.

In some embodiments, the tracking system includes one or more cameras.

In some embodiments, the tracking system includes one or more depth sensors.

In some embodiments, the tracking system is part of a head-mounted augmented reality surgical display system.

In some embodiments, each of the plurality of medical device fixation locations includes a fixation location of a pedicle screw, and wherein the implantable medical device includes a rod that can be shaped to be affixed to the plurality of medical device fixation locations.

According to some embodiments, a method of generating a shape of an implantable medical device includes: detecting, using a tracking system, a location of each of a plurality of markers that are each in a fixed relationship with respect to a medical device fixation location of a plurality of medical device fixation locations in a patient; determining a location of each of the medical device fixation locations based on the detected marker locations and the fixed relationships; generating a set of points in three-dimensional space that together define a curved line that passes through all of the determined locations of the medical device fixation locations; and outputting the generated set of points for use in shaping the implantable medical device.

In some embodiments, the set of points is generated during a surgical procedure that affixes the medical device fixation locations to the patient, and the set of points is generated based on the determined locations determined during the surgical procedure, not during preoperative planning.

In some embodiments, the tracking system includes one or more cameras.

In some embodiments, the tracking system includes one or more depth sensors.

In some embodiments, the tracking system is part of a head-mounted augmented reality surgical display system.

In some embodiments, each of the plurality of medical device fixation locations includes a fixation location of a pedicle screw, and wherein the implantable medical device includes a rod that can be shaped to be affixed to the plurality of medical device fixation locations.

For purposes of summarizing the disclosure, certain aspects, advantages, and novel features of embodiments of the disclosure have been described herein. It is to be understood that not necessarily all such advantages may be achieved in accordance with any particular embodiment of the disclosure disclosed herein. Thus, the embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein. The methods summarized above and set forth in further detail below describe certain actions taken by a practitioner; however, it should be understood that they can also include the instruction of those actions by another party. Thus, actions such as “detecting a location of each of a plurality of markers using a tracking system” include “instructing the detecting of a location of each of a plurality of markers using a tracking system.”

The disclosure will be more fully understood from the following detailed description of the embodiments thereof, taken together with the drawings.

### BRIEF DESCRIPTION OF THE DRAWINGS

FIG. 1 is a schematic pictorial illustration of a system for image-guided surgery, in accordance with an embodiment of the disclosure.

FIG. 2A is a schematic pictorial illustration of a head-mounted or near-eye unit for use in the system of FIG. 1.

FIG. 2B is a schematic pictorial illustration of another head-mounted or near-eye unit for use in the system of FIG. 1.

FIG. 3A is a schematic illustration of a side view of a spine having a plurality of pedicle screws and rods attached thereto.

FIG. 3B is a schematic illustration of a back view of the spine of FIG. 3A.

FIG. 4A is an embodiment of a flowchart depicting a process for guiding implantation of a medical device using an image-guided surgery system, such as the image-guided surgery system of FIG. 1.

FIG. 4B is another embodiment of a flowchart depicting another process for guiding implantation of a medical device using an image-guided surgery system, such as the image-guided surgery system of FIG. 1.

FIG. 4C is another embodiment of a flowchart depicting another process for guiding implantation of a medical device in an image-guided surgery system, such as the image-guided surgery system of FIG. 1.

FIG. 5 is an embodiment of a flowchart depicting a process for aligning a patient's vertebrae during a spinal fusion surgery and generating a bent rod for implantation in the patient.

FIGS. 6A-6D are embodiments of schematic diagrams illustrating a sequence of augmented reality overlays that may be depicted by, for example, the head-mounted or near-eye units of FIG. 2A or 2B during a spinal fusion surgery.

FIG. 7 is another embodiment of a schematic diagram illustrating an augmented reality overlay that may be depicted by, for example, the head-mounted or near-eye units of FIG. 2A or 2B during a spinal fusion surgery.

### DETAILED DESCRIPTION

The disclosure herein presents various embodiments of systems, methods, and devices for using augmented reality or image-guided surgery systems to guide implantation of medical devices. For example, the systems, methods, and devices disclosed herein may be used in spinal fusion surgery, among other surgical, medical, and/or diagnostic procedures or interventions. The surgical, medical, and/or diagnostic procedures may comprise open surgery, minimally-invasive surgery, endoscopic procedures, laparoscopic procedures, and/or the like.

In spinal surgery, such as in spinal fusion surgery, a surgeon will typically install one or more rods that mechanically connect two or more vertebrae or vertebral bodies together in order to prevent motion and allow fusion to occur across the disc space between the vertebral bodies. The rod is typically passed through the heads of pedicle screws that in turn are affixed to the vertebral bodies. In minimally invasive spinal fusion surgery, this task can be particularly time-consuming and tiring for the surgeon, increasing risk to the patient. This is because, among other things, the surgeon needs to blindly pass the rod through the heads of the screws. For example, in a typical minimally invasive spinal fusion surgery, the surgeon will first bend the rod in such a way to hold the spine in a desired configuration. This rod bending is often based on a preoperative plan and is designed to bring the spine into a correct position. After the rod is bent, a distal tip (alternatively referred to as distal end) of the rod is positioned at the first screw head, and then the surgeon slowly pushes it ahead and tries to guide it between the second and following screw heads. Because this process is performed blindly, or by feel, the process is time-consuming, tiring, and introduces risks to the patient.

Various embodiments disclosed herein address the above-identified problems by, among other things, facilitating guiding of the rod between the screw heads using an augmented reality surgical system. For example, an augmented reality surgical system may be used that, among other things, overlays an image aligned with reality, wherein the image includes a depiction of the spine and/or the pedicle screws attached thereto, the current position of the distal tip of the rod and/or at least a portion of the body of the rod, and one or more guidance attributes (e.g., navigational guides) that assist the surgeon in guiding the distal tip of the rod to the next pedicle screw. The embodiments disclosed herein include a number of benefits, including, for example, increasing accuracy of implantation of medical devices, reducing the time taken to do so, reducing risk to the patient, enabling completion of more complicated procedures that may otherwise be difficult or impossible to do in a minimally invasive fashion, and/or the like.

Various embodiments disclosed herein provide a number of features that assist in accomplishing the above-mentioned benefits. For example, some embodiments can calibrate and/or detect a position of the distal tip (or other portion) of the rod relative to a feature on or coupled to the proximal end of the rod, such as a handle the surgeon uses to push the rod forward. The system can then use that calibration to know where the distal tip of the rod is when it is within a minimally invasive surgical site (e.g., when it is not optically observable from outside the patient's body) by, for example, tracking the feature on or coupled to the proximal end of the rod during the surgical procedure. During the surgical procedure, the system can then use this data to provide guidance to the surgeon on how to move the rod during the implantation procedure. For example, a near-eye display may include one or more guidance attributes, such as an arrow indicative of a direction in which to move the rod (e.g., a direction in which to move the distal tip of the rod and/or the proximal end of the rod), a distance of the distal tip of the rod from the next screw head, and/or the like. The near-eye display may also be configured to visually display the distal tip of the rod overlaid over reality during the procedure.

In addition to just knowing the current position of the rod distal tip in the patient during a minimally invasive procedure, some embodiments disclosed herein can also generate a 3D model of the entire body of the rod and/or at least a portion of the body of the rod between the distal tip and the proximal end, and also display at least a portion of that 3D model on the near-eye display as the surgeon is performing the implantation procedure. As will be described in greater detail below, generation of such a 3D model can be accomplished in a number of ways, including, for example, tracing the bent rod with a calibrated instrument, such as a Jamshidi needle, using a depth sensor to detect the shape of the rod, using preoperative or intraoperative imaging, such as 3D CT or 2D fluoroscopy, and/or the like. Further, at least some of these techniques can enable generation of guidance attributes during the surgical procedure without requiring a marker on the handle coupled to the proximal end of the rod. That said, such techniques can also be used in combination with tracking a marker on the handle coupled to the proximal end of the rod.

Another feature provided by various embodiments disclosed herein is the ability to determine or generate a shape of a rod during a spinal fusion surgical procedure, and to automatically generate the shaped or bent rod during the surgical procedure, without the need for preoperative planning and designing of the shape of the rod before the surgical procedure. This can lead to more efficient, safer, and accurate surgical procedures. For example, in some embodiments, the system can be configured to detect the positions of each pedicle screw after the surgeon has installed the pedicle screws and aligned the vertebrae in a desired orientation. Based on those detected positions, the system can then be configured to generate a desired shape of a rod that will pass through the pedicle screws in the detected positions. In some embodiments, such generated shape can also be provided to a machine, such as a rod bending machine, in order to cause automatic shaping or bending of the rod during the surgical or other medical procedure.

### Example Surgical System

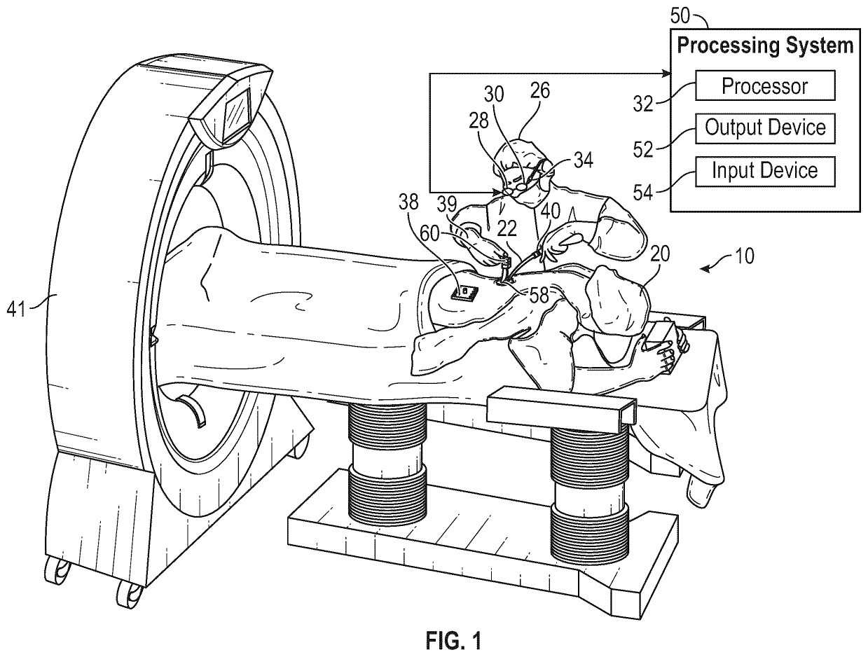

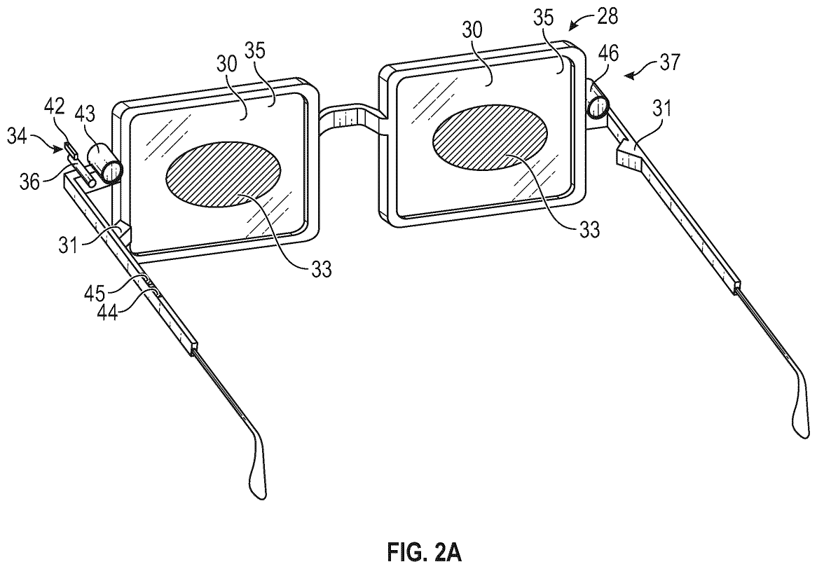

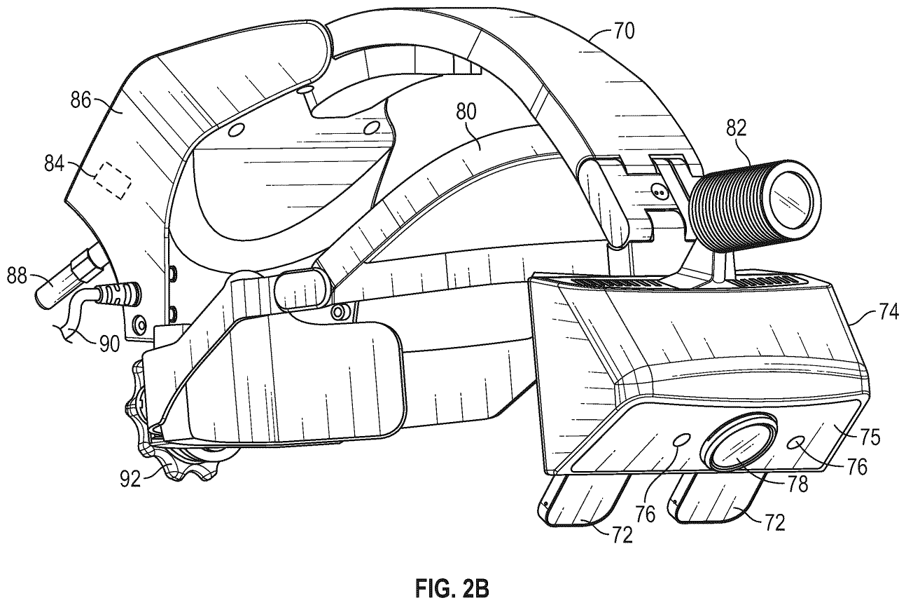

Reference is now made to FIGS. 1 and 2A, which schematically illustrate an exemplary system 10 for image-guided surgery, in accordance with some embodiments of the disclosure. For example, FIG. 1 is a pictorial illustration of the system 10 as a whole, while FIG. 2A is a pictorial illustration of a near-eye display unit that is used in the system 10. The near-eye display unit illustrated in FIGS. 1 and 2A is configured as a head-mounted display unit 28. In some embodiments, the near-eye display unit can be configured as a head-mounted AR display (HMD) unit 70, shown in FIG. 2B and described herein below. The head-mounted display unit may be in the form of glasses, spectacles, goggles, a visor, a head-up display, an over-the-head unit, a unit with a forehead strap, or other structure that facilitates an augmented reality display that can be viewed by a surgeon or other healthcare professional. In FIG. 1, the system 10 is applied in a medical procedure on a patient 20 using image-guided surgery. In this procedure, a tool 22 is inserted via an incision in the patient's back, in order to perform a surgical intervention. Alternatively, the system 10 and the techniques described herein may be used, mutatis mutandis, in other surgical procedures or in non-surgical procedures (e.g., minimally invasive, laparoscopic, or endoscopic medical treatment and/or diagnostic procedures).

Although not required for all techniques disclosed herein, at least some techniques disclosed herein can utilize depth sensing and/or depth mapping, and the example head-mounted display unit 28 includes features for conducting such depth sensing and/or depth mapping. Other embodiments, such as embodiments utilizing guiding techniques that do not require depth sensing and/or depth mapping, may use similar head-mounted display units that do not include depth sensing and/or depth mapping features.

Methods for optical depth mapping can generate a three-dimensional (3D) profile of the surface of a scene by processing optical radiation reflected from the scene. The terms depth map, 3D profile, and 3D image may be used interchangeably to refer to, for example, an electronic image in which the pixels contain values of depth or distance from a reference point, instead of or in addition to values of optical intensity.

In some embodiments, depth mapping or depth sensing systems can use structured light techniques in which a known pattern of illumination is projected onto the scene. Depth can be calculated based on the deformation of the pattern in an image of the scene. In some embodiments, depth mapping systems use stereoscopic techniques, in which the parallax shift between two images captured at different locations is used to measure depth. In some embodiments, depth mapping systems can sense the times of flight of photons to and from points in the scene in order to measure the depth coordinates. In some embodiments, depth mapping systems control illumination and/or focus and can use various sorts of image processing techniques.

In the embodiment illustrated in FIG. 1, a user of the system 10, such as a healthcare professional 26 (for example, a surgeon performing the procedure), wears the head-mounted display unit 28. In some embodiments, the head-mounted display unit 28 includes one or more see-through displays 30, for example as described in the above-mentioned U.S. Pat. No. 9,928,629 or in the other patents and applications cited above.

In some embodiments, the one or more see-through displays 30 include an optical combiner. In some embodiments, the optical combiner is controlled by one or more processors 32. In some embodiments, the one or more processors 32 are disposed in a central processing system 50. In some embodiments, the one or more processors 32 are disposed in the head-mounted display unit 28. In some embodiments, the one or more processors 32 are disposed in both the central processing system 50 and the head-mounted display unit 28 and can share processing tasks and/or allocate processing tasks between the one or more processors 32.

In some embodiments, the one or more see-through displays 30 display an augmented-reality image to the healthcare professional 26. In some embodiments, the augmented reality image viewable through the one or more see-through displays 30 is a combination of objects visible in the real world with the computer-generated image. In some embodiments, each of the one or more see-through displays 30 comprises a first portion 33 and a second portion 35. In some embodiments, the one or more see-through displays 30 display the augmented-reality image such that the computer-generated image is projected onto the first portion 33 in alignment with the anatomy of the body of the patient 20 that is visible to the healthcare professional 26 through the second portion 35. Portion 33 may be transparent (see-through), substantially transparent, semi-transparent, opaque, substantially opaque, or semi-opaque.

In some embodiments, the computer-generated image includes an image of the patient (e.g., patient 20) anatomy, such as an X-ray image, CT image, or MRI image. In some embodiments the anatomy image may be aligned with the anatomy of the patient. In some embodiments, all or a portion of the imaged anatomy may not be visible to the healthcare professional 26. For example, in a minimally invasive spine surgery, a CT image of the patient's spine may be projected onto portion 33 of see-through displays 30 and overlaid or augmented on the skin of the back of patient 20 while aligned with the anatomy of patient 20. In some embodiments, the computer-generated image includes a virtual image of one or more tools 22. In some embodiments, the system 10 combines at least a portion of the virtual image of the one or more tools 22 into the computer-generated image. In some embodiments, at least a portion of the virtual image of the one or more tools 22 is overlaid or augmented on an image of patient 20 anatomy and in alignment with the image of the anatomy. For example, some or all of the tool 22 may not be visible to the healthcare professional 26 because, for example, a portion of the tool 22 is hidden by the patient's anatomy (e.g., a distal end of the tool 22). In some embodiments, the system 10 can display the virtual image of at least the hidden portion of the tool 22 as part of the computer-generated image displayed in the first portion 33. In this way, the virtual image of the hidden portion of the tool 22 is displayed on the patient's anatomy. In some embodiments, the portion of the tool 22 hidden by the patient's anatomy increases and/or decreases over time or during the procedure. In some embodiments, the system 10 increases and/or decreases the portion of the tool 22 included in the computer-generated image based on the changes in the portion of the tool 22 hidden by the patient's anatomy over time.

Some embodiments of the system 10 comprise an anchoring device (e.g., bone marker or anchoring device 60), such as a clamp or a pin, for indicating the body of the patient 20. For example, in image-guided surgery and other surgeries that utilize the system 10, the bone marker 60 can be used as a fiducial marker. In some embodiments, the bone marker 60 can be coupled with the fiducial marker. In system 10, for example, the anchoring device is configured as the bone marker 60 (e.g., anchoring device coupled with a marker that is used to register an ROI of the body of the patient 20). In some embodiments, patient 20 anatomy is registered with the anchoring device and with a tracking system, via a preoperative or intraoperative CT scan of the ROI. A registration marker coupled with or included in bone marker or anchoring device 60 and/or a marker 38 may be utilized in such a registration procedure. During the procedure, in some embodiments, the tracking system (for example, an IR tracking system) tracks the marker mounted on the anchoring device and the tool 22 mounted with a tool marker 40. Following that, the display of the CT image data, including, for example, a model generated based on such data, on the near-eye display may be aligned with the surgeon's actual view of the ROI based on this registration. In addition, a virtual image of the tool 22 may be displayed on the CT model based on the tracking data and the registration. The surgeon or other healthcare professional may then navigate the tool 22 based on the virtual display of the tool 22 with respect to the patient image data, and optionally, while it is aligned with the user view (e.g., view of the surgeon or other healthcare professional wearing a head-mounted display unit) of the patient or ROI.

According to some aspects, the image presented on the one or more see-through displays 30 is aligned with the body of the patient 20. According to some aspects, misalignment of the image presented on the one or more see-through displays 30 with the body of the patient 20 may be allowed. In some embodiments, the misalignment may be 0-1 mm, 1-2 mm, 2-3 mm, 3-4 mm, 4-5 mm, 5-6 mm, and overlapping ranges therein. According to some aspects, the misalignment may typically not be more than about 5 mm. In order to account for such a limit on the misalignment of the patient's anatomy with the presented images, the position of the patient's body, or a portion thereof, with respect to the head-mounted display unit 28 can be tracked. For example, in some embodiments, a marker 38 and/or the bone marker 60 attached to an anchoring implement or device, such as a clamp 58 or pin, for example, may be used for this purpose, as described further hereinbelow.

When an image of the tool 22 is incorporated into the computer-generated image that is displayed on the head-mounted display unit 28 or the IMD unit 70, the position of the tool 22 with respect to the patient's anatomy should be accurately reflected. For this purpose, the position of the tool 22 or a portion thereof, such as the tool marker 40, is tracked by the system 10. In some embodiments, the system 10 determines the location of the tool 22 with respect to the patient's body such that errors in the determined location of the tool 22 with respect to the patient's body are reduced. For example, in certain embodiments, the errors may be 0-1 mm, 1-2 mm, 2-3 mm, 3-4 mm, 4-5 mm, and overlapping ranges therein.

In some embodiments, the head-mounted unit 28 includes a tracking sensor 34 to facilitate determination of the location and orientation of the head-mounted display unit 28 with respect to the patient's body and/or with respect to the tool 22. In some embodiments, tracking sensor 34 can also be used in finding the position and orientation of the tool 22 and professional 26 with respect to the patient's body. In some embodiments, the tracking sensor 34 comprises an image-capturing device 36, such as a camera, which captures images of the marker 38, the bone marker 60, and/or the tool marker 40. For some applications, an inertial-measurement unit 44 is also disposed on the head-mounted display unit 28 to sense movement of the surgeon or other healthcare professional's head.

In some embodiments, the tracking sensor 34 includes a light source 42. In some embodiments, the light source 42 is mounted on the head-mounted display unit 28. In some embodiments, the light source 42 irradiates the field of view of the image-capturing device 36 such that light reflects from the marker 38, the bone marker 60, and/or the tool marker 40 toward the image-capturing device 36. In some embodiments, the image-capturing device 36 comprises a monochrome camera with a filter that passes only light in the wavelength band of light source 42. For example, the light source 42 may be an infrared light source, and the camera may include a corresponding infrared filter. In some embodiments, the marker 38, the bone marker 60, and/or the tool marker 40 comprise patterns that enable a processor to compute their respective positions, i.e., their locations and their angular orientations, based on the appearance of the patterns in images captured by the image-capturing device 36. Suitable designs of these markers and methods for computing their positions and orientations are described in the patents and patent applications incorporated herein and cited above.

In addition to or instead of the tracking sensor 34, the head-mounted display unit 28 can include a depth sensor 37. In the embodiment shown in FIG. 2A, the depth sensor 37 comprises a light source 46 and a camera 43. Camera 43 may include one or more cameras, e.g., two cameras, which may be mounted on HMD 28 in various layouts. In some embodiments, the light source 46 projects a pattern of structured light onto the region of interest (ROI) that is viewed through the one or more displays 30 by a user, such as professional 26, who is wearing the head-mounted display unit 28. The camera 43 can capture an image of the pattern on the ROI and output the resulting depth data to the processor 32 and/or processor 45. The depth data may comprise, for example, either raw image data or disparity values indicating the distortion of the pattern due to the varying depth of the ROI. In some embodiments, the processor 32 computes a depth map of the ROI based on the depth data generated by the camera 43.

In some embodiments, the camera 43 also captures and outputs image data with respect to the markers in system 10, such as marker 38, bone marker 60, and/or tool marker 40. In this case, the camera 43 may also serve as a part of tracking sensor 34, and a separate image-capturing device 36 may not be needed. For example, the processor 32 may identify marker 38, bone marker 60, and/or tool marker 40 in the images captured by camera 43. The processor 32 may also find the 3D coordinates of the markers in the depth map of the ROI. Based on these 3D coordinates, the processor 32 is able to calculate the relative positions of the markers, for example in finding the position of the tool 22 relative to the body of the patient 20, and can use this information in generating and updating the images presented on head-mounted display unit 28.

In some embodiments, the depth sensor 37 may apply other depth mapping technologies in generating the depth data. For example, the light source 46 may output pulsed or time-modulated light, and the camera 43 may be modified or replaced by a time-sensitive detector or detector array to measure the time of flight of the light to and from points in the ROI. As another option, the light source 46 may be replaced by another camera, and the processor 32 may compare the resulting images to those captured by the camera 43 in order to perform stereoscopic depth mapping. These and all other suitable alternative depth mapping technologies are considered to be within the scope of the present disclosure.

In the pictured embodiment, system 10 also includes a tomographic imaging device, such as an intraoperative computerized tomography (CT) scanner 41. Alternatively or additionally, processing system 50 may access or otherwise receive tomographic data from other sources; and the CT scanner itself is not an essential part of the present system. In some embodiments, regardless of the source of the tomographic data, the processor 32 can compute a transformation over the ROI so as to register the tomographic images with the depth maps that it computes on the basis of the depth data provided by depth sensor 37. The processor 32 can then apply this transformation in presenting a part of the tomographic image on the one or more displays 30 in registration with the ROI viewed through the one or more displays 30.

In some embodiments, in order to generate and present an augmented reality image on the one or more displays 30, the processor 32 computes the location and orientation of the head-mounted display unit 28 with respect to a portion of the body of patient 20, such as the patient's back. In some embodiments, the processor 32 also computes the location and orientation of the tool 22 with respect to the patient's body. In some embodiments, the processor 45, which can be integrated within the head-mounted display unit 28, may perform these functions. Alternatively or additionally, the processor 32, which is disposed externally to the head-mounted display unit 28 and can be in wireless communication with the head-mounted display unit 28, may be used to perform these functions. The processor 32 can be part of the processing system 50, which can include an output device 52, for example a display, such as a monitor, for outputting information to an operator of the system, and/or an input device 54, such as a pointing device, a keyboard, a foot pedal, or a mouse, to allow the operator to input data into the system. In some embodiments HMD 28 may include one or more input devices, such as a touch screen or buttons.

Alternatively or additionally, users of the system 10 (e.g., surgeons or other healthcare professionals 26) may input instructions to the processing system 50 using a gesture-based interface. For this purpose, for example, the depth sensor 37 may sense movements of a hand 39 of the healthcare professional 26. Different movements of the professional's hand and fingers may be used to invoke specific functions of the one or more displays 30 and of the system 10.

In general, in the context of the present description, when a computer processor is described as performing certain steps, these steps may be performed by external computer processor 32 and/or computer processor 45 that is integrated within the head-mounted unit. The processor or processors carry out the described functionality under the control of suitable software, which may be downloaded to system 10 in electronic form, for example over a network, and/or stored on tangible, non-transitory computer-readable media, such as electronic, magnetic, or optical memory.

FIG. 2B is a schematic pictorial illustration showing details of a head-mounted AR display (HMD) unit 70, according to another embodiment of the disclosure. HMD unit 70 may be worn by the healthcare professional 26, and may be used in place of the head-mounted display unit 28 (FIG. 1). HMD unit 70 comprises an optics housing 74 which incorporates a camera 78, and in the specific embodiment shown, an infrared camera. In some embodiments, the housing 74 comprises an infrared-transparent window 75, and within the housing (e.g., behind the window) are mounted one or more, for example two, infrared projectors 76. One of the infrared projectors and the camera may be used, for example, in implementing a pattern-based depth sensor.

In some embodiments, mounted on housing 74 are a pair of augmented reality displays 72, which allow professional (e.g., surgeon) 26 to view entities, such as part or all of patient 20, through the displays, and which are also configured to present to professional 26 images or any other information. In some embodiments, the displays 72 present planning and guidance information, as described above.

In some embodiments, the HMD unit 70 includes a processor 84, mounted in a processor housing 86, which operates elements of the HMD unit. In some embodiments, an antenna 88 may be used for communication, for example with processor 32 (FIG. 1).

In some embodiments, a flashlight 82 may be mounted on the front of HMD unit 70. In some embodiments, the flashlight may project visible light onto objects so that the professional 26 is able to clearly see the objects through displays 72. In some embodiments, elements of the HMD unit 70 are powered by a battery (not shown in the figure), which supplies power to the elements via a battery cable input 90.

In some embodiments, the HMD unit 70 is held in place on the head of professional 26 by a head strap 80, and the professional may adjust the head strap by an adjustment knob 92.

In some embodiments, the HMD may comprise a visor, which includes an AR display positioned in front of each eye of the professional and controlled by the optical engine to project AR images into the pupil of the eye.

In some embodiments, the HMD may comprise a light source for tracking applications, comprising, for example, a pair of infrared (IR) LED projectors, configured to direct IR beams toward the body of patient 20. In some embodiments, the light source may comprise any other suitable type of one or more light sources, configured to direct any suitable wavelength or band of wavelengths of light. The HMD may also comprise one or more cameras, for example, a red/green/blue (RGB) camera having an IR-pass filter, or a monochrome camera configured to operate in the IR wavelengths. The one or more cameras may be configured to capture images including the markers in system 10 (FIG. 1).

In some embodiments, the HMD may also comprise one or more additional cameras, e.g., a pair of RGB cameras. In some embodiments, each RGB camera may be configured to produce high-resolution RGB (HR RGB) images of the patient's body, which can be presented on the AR displays. Because the RGB cameras are positioned at a known distance from one another, the processor can combine the images to produce a stereoscopic 3D image of the site being operated on.

In some embodiments, it may be possible to 3D reconstruct any shape from a pair of stereo cameras (e.g., left and right cameras with known relative rigid translation and rotation). For example, implants, navigation tools, or other objects could be modeled and reconstructed in 3D by capturing left and right images of the same object and determining the pixel corresponding to the same object within the left and right images. In some embodiments, the determined correspondences plus the calibration data (e.g., the cameras' relative transformation) advantageously make it feasible to 3D reconstruct any object. In accordance with several implementations, in order to fully 3D reconstruct an object, the depth sensing systems could capture left and right images of the object from multiple angles or views, with each angle or view providing a partial 3D point cloud of an implant, instrument, tool, or other object. The images from multiple angles or views (e.g., and associated respective partial 3D point clouds) could be combined or stitched together. After modeling an object, the systems could calibrate the object into one of the reference markers described herein.

In some embodiments, the HMD light source 46 (FIG. 2A) may comprise a structured light projector (SLP) which projects a pattern onto an area of the body of patient 20 on which professional 26 is operating. In some embodiments, light source 46 comprises a laser dot pattern projector, which is configured to apply to the area structured light comprising a large number (typically between hundreds and hundreds of thousands) of dots arranged in a suitable pattern. This pattern serves as an artificial texture for identifying positions on large anatomical structures lacking fine details of their own, such as the skin and surfaces of the vertebrae. In some embodiments, one or more cameras 43 capture images of the pattern, and a processor, such as processor 32 (FIG. 1), processes the images in order to produce a depth map of the area. In some embodiments, the depth map is calculated based on the local disparity of the images of the pattern relative to an undistorted reference pattern, together with the known offset between the light source and the camera. The artificial texture added by the structured light sensor could provide for improved detection of corresponding pixels between left and right images obtained by left and right cameras. In some embodiments, the structured light sensor could act as a camera, such that instead of using two cameras and a projector, depth sensing and 3D reconstruction may be provided using only a structured light sensor and a single camera.

In some embodiments, the projected pattern comprises a pseudorandom pattern of dots. In this case, clusters of dots can be uniquely identified and used for disparity measurements. In the present example, the disparity measurements may be used for calculating depth and for enhancing the precision of the 3D imaging of the area of the patient's body. In some embodiments, the wavelength of the pattern may be in the visible or the infrared range.

In some embodiments, the system 10 (FIG. 1) may comprise a structured light projector (not shown) mounted on a wall or on an arm of the operating room. In such embodiments, a calibration process between the structured light projector and one or more cameras on the head-mounted unit or elsewhere in the operating room may be performed to obtain the 3D map.

### Example Spinal Surgery Elements

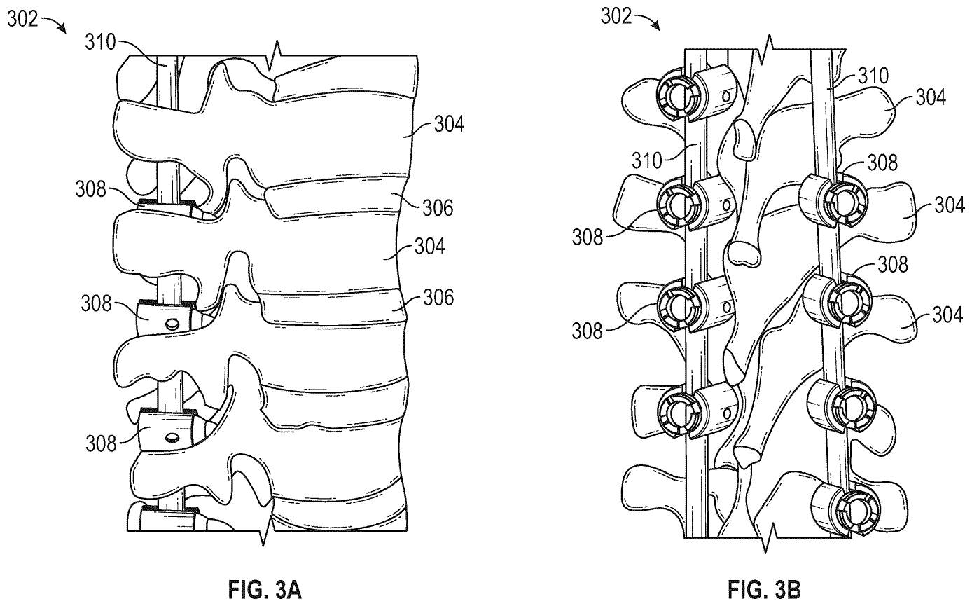

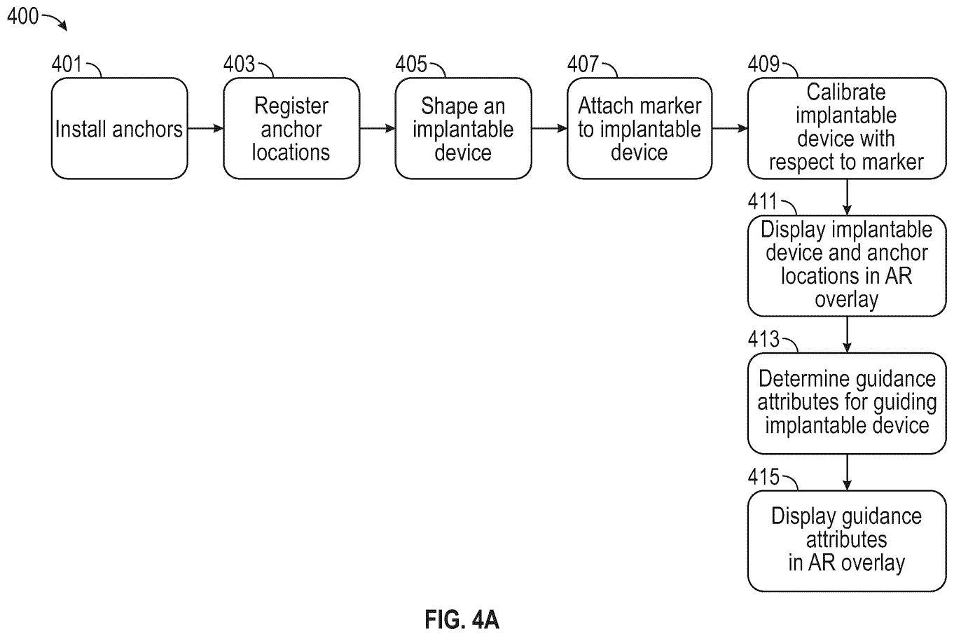

FIGS. 3A and 3B illustrate schematically a number of elements relevant to spinal surgery, such as spinal fusion surgery. FIG. 3A is a side view, and FIG. 3B is a back view, or rear view. Each of these figures depicts a portion of a patient's spine 302 that comprises a plurality of vertebrae 304 separated by intervertebral discs 306. These figures also show a plurality of pedicle screws 308 having been implanted into the vertebrae 304. Two rods 310 have also been passed through the pedicle screws 308 and affixed thereto, thus affixing the adjacent vertebrae 304 to one another, to facilitate spinal fusion.

### Example Implantable Device Guidance Process

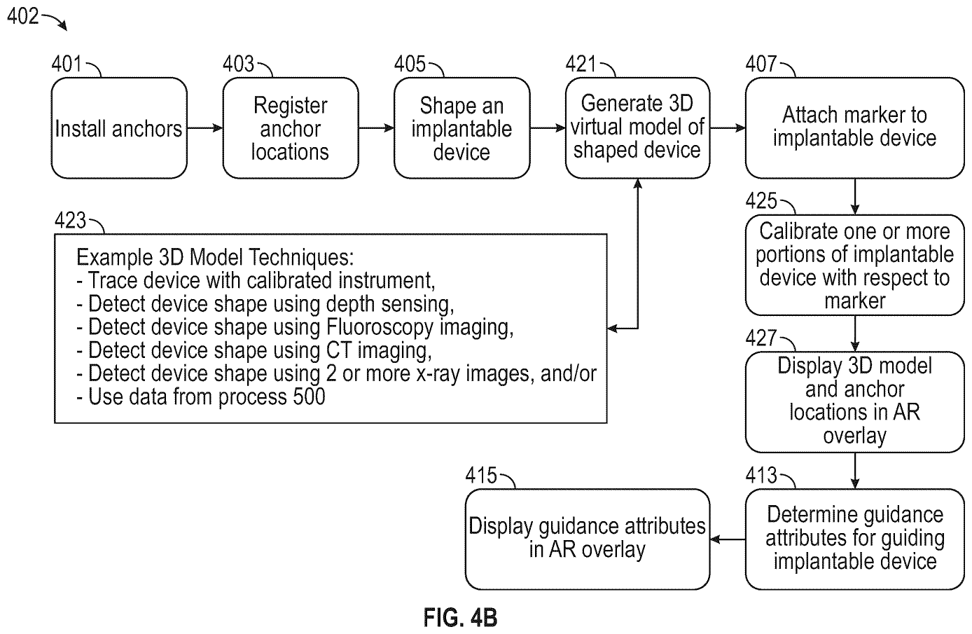

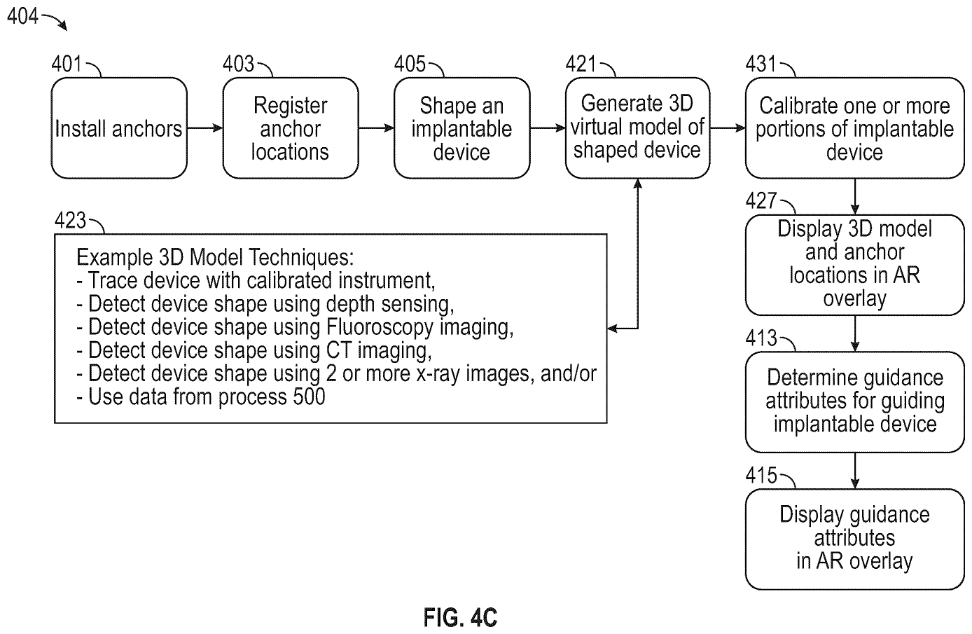

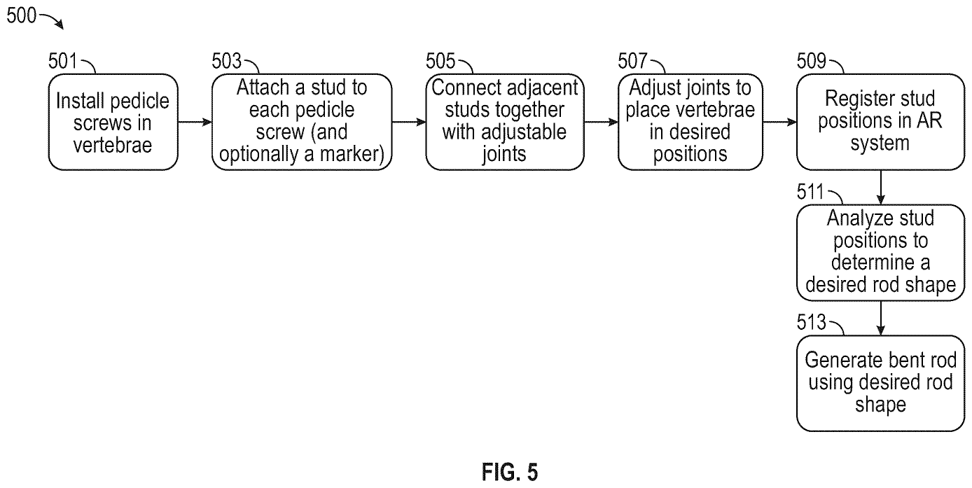

FIG. 4A illustrates an example embodiment of a flowchart illustrating a process 400 for guiding a portion of an implantable device during a surgical procedure (such as, for example, guiding the distal tip of a rod during a spinal fusion surgery). At block 401, a plurality of anchors are installed. For example, a plurality of pedicle screws 308 may be installed in vertebrae 304, as shown in FIGS. 3A and 3B. The pedicle screws 308 may act as anchors for a medical device or implant, such as a rod 310 of FIGS. 3A and 3B, to be coupled thereto.

At block 403, locations of the installed anchors are registered. For example, the head-mounted unit 28, 70 may be configured to detect the locations of the heads of the pedicle screws installed at block 401, and store their locations in a memory of the augmented reality surgical system. In some embodiments, the system may detect the locations of the screw heads in reference to a reference point, such as the marker 38 and/or 60 of FIG. 1. The detection or registration of locations of the anchors, such as the pedicle screw heads, may be accomplished using a variety of processes, including, but not limited to, the processes described above and in any of the patents and publications referenced above. As noted below, in some embodiments, the locations of the anchors may be predetermined during preoperative planning, and installation of the anchors may be guided by the augmented reality surgical system. In some such embodiments, block 403 may not occur, since the system is already aware of the planned anchor locations. In some such embodiments, however, block 403 may still occur, such as to account for any differences between the planned anchor locations and the actual installed anchor locations.

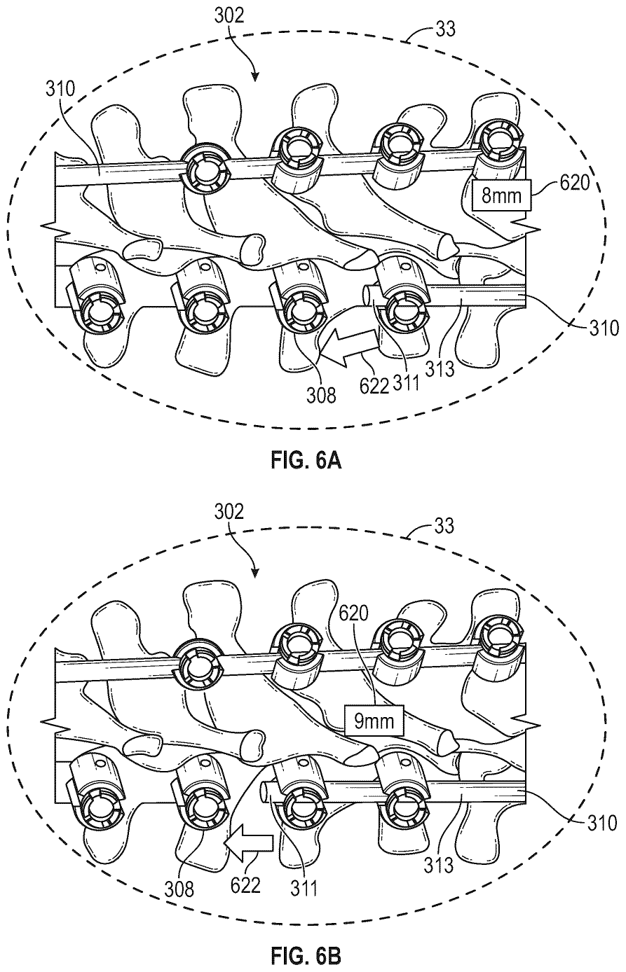

At block 405, after the anchor or pedicle screw head locations are known, an implantable device, such as a rod 310 of FIG. 3B, may be shaped into a desired shape. For example, a rod 310 may be bent into a shape that will pass through the registered pedicle screw head locations and result in positioning the patient's vertebrae in the desired configuration. In some embodiments, shaping of the rod 310 may be conducted manually, or may be conducted automatically by a machine. In some embodiments, shaping of the rod 310 may be conducted using the process 500 described below with reference to FIG. 5. Further, in some embodiments, shaping of the rod 310 may occur prior to the surgical procedure, such as using preoperative CT imaging or fluoroscopy imaging, or another type of imaging scan. Positions of the pedicle screws may also be determined during such preoperative planning, and installation of the pedicle screws may be guided by the augmented reality system, to help ensure that the final pedicle screw locations match the planned locations (or are sufficiently close to the planned locations).

Next, at block 407, a marker is attached to the implantable device. For example, in the context of a spinal fusion surgery, a handle may be attached to a proximal end of a rod 310, and the handle may have a marker coupled thereto (e.g., tool 22 and marker 40 of FIG. 1). At block 409, the implantable device may be calibrated with respect to the marker attached at block 407. For example, the system may be configured to detect a relationship between the distal tip of the rod 310 with respect to the marker coupled to the handle that is coupled to the proximal end of the rod 310. By knowing a relationship between the distal tip of the rod 310 and the marker coupled to the handle, the augmented reality system can determine where the distal tip of the rod 310 is at any time during the surgical procedure, even if the distal tip is within the patient's body and not optically visible from outside of the patient's body. Examples of techniques for calibrating a portion of a medical device with respect to the handle marker, tracking the handle marker during a surgical procedure, and determining the location of the portion of the medical device during the surgical procedure based on the tracked location of the handle marker can be found in, for example, one or more of the patents and publications referenced above. In some embodiments, the proximal end of the rod itself may incorporate a marker and/or the proximal end of the rod may have a particular shape, configuration, mechanical form, and/or the like that has a unique appearance from various viewpoints (or from any viewpoint), such that the proximal end of the rod can be tracked by the augmented reality surgical system, and the augmented reality surgical system can determine the position and/or orientation of the rod in 3D without having to calibrate the rod to a separate marker. In such an embodiment, blocks 407 and 409 may not be included, although they may still be included in some such embodiments, such as for redundancy and/or increased accuracy.

At block 411, during the surgical procedure, the system can display at least a portion of the implantable device and at least a portion of the anchor locations in an augmented reality overlay. For example, a see-through display of the head-mounted unit 28, 70 may overlay an image over reality that shows, for example, a position of the distal tip of the rod 310 and one or more of the pedicle screw 308 positions.

At block 413, the system can determine guidance attributes (e.g., navigational guides) for guiding the implantable device. For example, the system can be configured to determine guidance attributes for guiding the distal tip of the rod 310 to the next pedicle screw 308. Example guidance attributes may include, for example, an arrow, angle, and/or other directional indicator that indicates a direction in which to move the distal tip and/or the proximal end of the rod 310, a distance that the distal tip of the rod 310 is away from the next pedicle screw 308, and/or the like.

Various methods may be used to determine the guidance attributes. In some embodiments, guidance attributes may be determined based on a preoperative plan. For example, a preoperative plan may indicate the desired final positioning of the implantable device and/or a planned trajectory of the implantable device to achieve the desired final positioning, and guidance attributes may be determined based on, for example, a current deviation of a position or orientation of the implantable device from the desired final position and/or planned trajectory. Determining guidance attributes based only on a preoperative plan can have some downsides, however, in accordance with some embodiments. For example, the actual position and orientation of the anchors installed at block 401 and/or the actual shape of the implantable device shaped at block 405 may often deviate somewhat from the preoperative plan. Accordingly, in some embodiments, the guidance attributes determined at block 413 are determined in real time during the surgical procedure based on the presently detected or tracked positions and orientations of the implantable device and/or of one or more of the anchors without reliance on a preoperative plan. These presently detected or tracked positions may be determined through direct or indirect tracking (such as, for example, directly tracking the distal tip of an exposed rod, directly tracking an exposed anchor, indirectly tracking the distal tip of a rod via a marker coupled to the rod, indirectly tracking an anchor via a patient or bone marker, and/or the like).

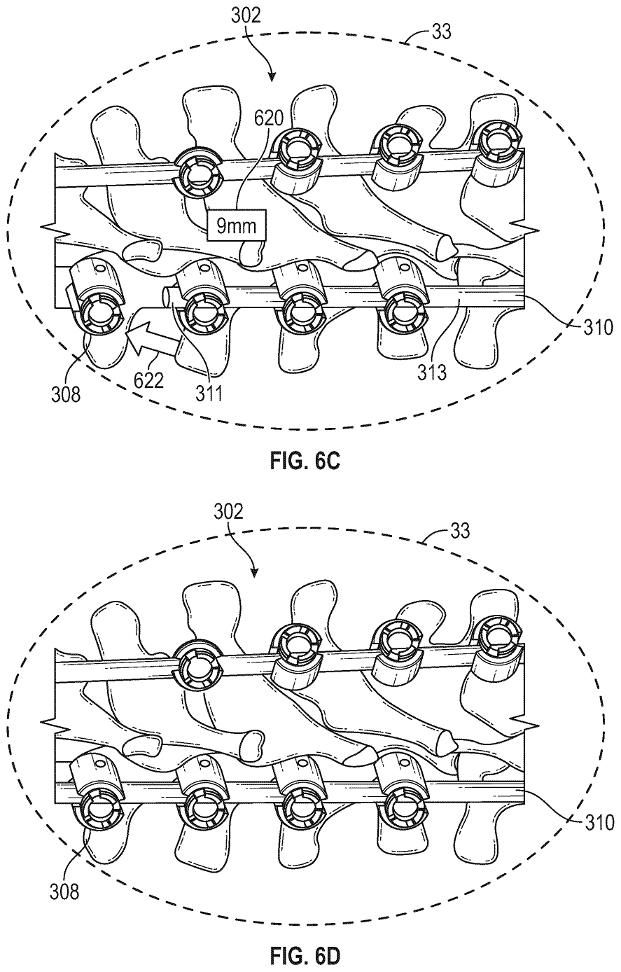

For example, as further discussed below with reference to FIGS. 6A-6C, the system may be configured to determine the guidance attributes (such as, for example, navigation guides 620 and/or 622) based on the presently detected or tracked position and/or orientation of the rod 310 and/or the distal tip 311, and where the distal tip 311 currently is in relation to the next anchor (e.g., pedicle screw 308). In some embodiments, the guidance attributes may be determined based on the presently detected or tracked positions and orientations of the implantable device and one or more of the anchors, in combination with a preoperative plan. For example, the guidance attributes may additionally take into account a deviation from a planned trajectory. However, in some embodiments, the guidance attributes may be determined based on the presently detected or tracked (directly or indirectly) positions and orientations of the implantable device and one or more of the anchors without referencing a preoperative plan and/or combining such information with a preoperative plan. In other words, the system may be configured to generate guidance attributes based on how the surgeon should manipulate the implantable device in order to advance the implantable device to the next anchor (e.g., to the next closest anchor of a plurality of anchors), using the current real-time tracking of the implantable device's position and orientation and/or the current real-time tracking of the patient's and/or the anchor's position and orientation, as opposed to calculating a deviation of the implantable device's position or orientation from a preplanned path. Stated another way, the system may be configured to determine guidance attributes based on a deviation between the present location of a portion of the implantable device (such as the distal tip 311 shown in FIG. 6A) and the present location of the next anchor (such as the pedicle screw 308 of FIG. 6A), without requiring or referencing a preplanned path. Such configurations may be desirable, for example, to increase efficiency, increase accuracy, reduce preoperative planning requirements, reduce the overall time required to conduct a surgical intervention, reduce computing requirements during the surgical intervention, reduce latency in the augmented reality display, and/or the like.

With continued reference to FIG. 4A, next, at block 415, the guidance attributes determined at block 413 are displayed in the augmented reality overlay. For example, the see-through display of head-mounted unit 28, 70 may be configured to display one or more of the guidance attributes (such as, for example, one or more arrows, distances, and/or the like) in order to help the surgeon guide the distal tip of the rod 310 to each screw head of the pedicle screws 308. Examples of this are also shown in FIGS. 6A-6C, described below.

### Additional Example Implantable Device Guidance Process

FIG. 4B illustrates another example embodiment of a flowchart illustrating a process 402 for guiding implantation of an implantable device. The process 402 has many similarities to the process 400 of FIG. 4A, and the same or similar reference numbers are used to refer to the same or similar blocks. For example, at blocks 401, 403, and 405, anchors are installed, locations of the anchors are registered in the system, and an implantable device is shaped, as described above with reference to process 400.

One difference in the process 402 is that this process is capable of generating a 3D virtual model of an implantable device, and displaying at least a portion of that 3D virtual model in the see-through display of an augmented reality surgical system, such as in a see-through display of the head-mounted unit 28, 70. This can be beneficial, for example, because it can, among other things, allow the surgeon to see the entire or at least a portion of the body of the rod 310 while implanting the rod into the patient, instead of just seeing a depiction of the distal tip of the rod 310.

With continued reference to FIG. 4B, at block 421, the system generates a 3D virtual model of a shaped device, such as the implantable device that was shaped or bent at block 405. Generation of the 3D virtual model can be accomplished using a number of techniques. A nonexclusive list of such techniques is provided in block 423. For example, one technique is to trace the shaped device (e.g., a bent rod 310 for spinal fusion surgery) with a calibrated instrument. For example, the augmented reality surgical system may calibrate a surgical instrument, such as a Jamshidi needle, such that movement of the Jamshidi needle can be tracked by the system, and the surgeon or other healthcare professional can then trace the bent rod with a tip of the Jamshidi needle in order for the system to detect the shape of the bent rod.

Another example technique for generating the 3D virtual model is to detect the shape of the bent rod using the depth sensor 37 of, for example, head-mounted unit 28, 70. Examples of using such a depth sensor to extract information about the environment or a device, such as the bent rod, are provided in PCT Publication No. WO 2023/021448, titled AUGMENTED-REALITY SURGICAL SYSTEM USING DEPTH SENSING, published Feb. 23, 2023, and in Appendix A of U.S. Provisional Application No. 63/520,215, filed Aug. 17, 2023, titled AUGMENTED REALITY NAVIGATION BASED ON MEDICAL IMPLANTS, (which corresponds to U.S. Provisional Application No. 63/447,368, titled AUGMENTED-REALITY SURGICAL SYSTEM USING DEPTH SENSING, filed Feb. 22, 2023), both of which are incorporated by reference herein in their entirety. Depth sensing can have a number of benefits, including facilitating, for example, calibration of non-straight instruments, haptic feedback, reduced effects of patient breathing on accuracy, occlusion capabilities, gesture recognition, 3D reconstruction of any shape or object, monitoring and quantifying of removed volumes of tissue, implant modeling and registration without reliance on X-rays.

Another example technique for generating the 3D virtual model is to detect the shape using inter-operative imaging, such as fluoroscopy imaging, CT imaging, and/or two or more X-ray images. Finally, another example technique for generating the 3D virtual model is to use data output from the process 500 of FIG. 5, described in further detail below.

With continued reference to FIG. 4B, at block 407, a marker may be attached to the implantable device, similar to as discussed above with reference to process 400. At block 425, one or more portions of the implantable device are calibrated with respect to the marker attached at block 407. This block can be similar to block 409 of process 400, discussed above, which included, for example, calibrating the distal tip of a rod 310 with respect to the marker applied to a handle attached to a proximal end of the rod 310. One difference, however, is that since process 402 includes a 3D virtual model of a body of the implantable device, such as rod 310, the entire body or at least a portion of the body of the implantable device may also be calibrated in the system with respect to the marker. Accordingly, the augmented reality system, such as including the head-mounted unit 28, 70, may be able to track and display not only the tip of the rod 310 during a surgical procedure, but also all of the body or a portion of the body of the rod 310 between the distal tip and the handle attached to the proximal end of the rod 310.

At block 427, the augmented reality system, such as a see-through display of the head-mounted unit 28, 70, may display the 3D model, or at least a portion of the 3D model, and one or more of the anchor locations in an augmented reality overlay. This may be similar to block 411 of process 400, except that at least a portion of the 3D model of the body of the implantable device is displayed in the AR overlay in addition to or in lieu of displaying only the distal tip of rod 310.

At blocks 413 and 415, the process flow proceeds similarly to as for the same blocks of process 400 discussed above. Namely, the system can determine one or more guidance attributes for guiding implantation of the implantable device and display one or more of those determined guidance attributes in the AR overlay during the surgical procedure.

### Additional Example Implantable Device Guidance Process

FIG. 4C illustrates another embodiment of a flowchart depicting a process 404 for guiding implantation of a medical device, such as a rod used in spinal fusion surgery. The process 404 has many similarities to the process 402 discussed above, and the same or similar blocks utilize the same or similar reference numbers. One difference from the process 402, however, is that the process 404 depicts a process that can enable guiding of implantation of a medical device without requiring a marker (such as a marker on a handle attached to a proximal end of a rod 310). That said, the process depicted in FIG. 4C may also be used with other processes disclosed herein that use a marker, such as for redundancy and/or increased accuracy.

Blocks 401, 403, 405, and 421 proceed the same as described above with reference to process 402. At block 431, however, once a 3D virtual model of the shaped device (e.g., a bent rod such as a rod 310 for use in spinal fusion surgery) has been generated, the shaped implantable device may be calibrated directly without requiring use of a marker coupled thereto (such as a marker on a handle coupled to a proximal end of the rod 310). For example, the depth sensor 37 of head-mounted unit 28, 70 may be used before and/or during the surgical procedure to detect and track the location and orientation of the shaped device, such as of the bent rod 310, with respect to the patient and/or with respect to a location that is fixed with respect to the patient, such as marker 38 and/or 60. The detection and tracking using the depth sensor 37 may be conducted similarly as described above with respect to generating a 3D virtual model of the bent rod 310 using the depth sensor 37, and utilizing any of the techniques disclosed in PCT Publication No. WO 2023/021448, or Appendix A of U.S. Provisional Application No. 63/520,215 (corresponding to U.S. Provisional Application No. 63/447,368).

The process flow then proceeds to blocks 427, 413, and 415, wherein the process operates similarly to as described above with reference to processes 400 and 402.

### Example Spinal Alignment and Shaped Rod Generation Process

FIG. 5 illustrates an embodiment of a flowchart depicting a process 500 for aligning vertebrae and generating a bent rod for use in spinal fusion surgery during the surgical procedure. One benefit of the process 500 illustrated in FIG. 5 is that one or more rods for use in the spinal fusion surgical procedure may be generated on demand during the surgical procedure, instead of based on preoperative planning. Such a process may result in a more accurate, safer, and/or efficient and time-saving procedure.

At block 501, a plurality of pedicle screws are installed in vertebrae. For example, the various pedicle screws 308 may be installed in vertebrae 304, as shown in FIGS. 3A and 3B. At block 503, a stud may be attached to each of the installed pedicle screws. For example, each of the studs may comprise metal, stainless steel, titanium, and/or the like. Each stud may also comprise a known geometry, and may comprise a marker, an interface for connecting a marker, a reflective sphere, and/or the like. If the studs comprise an interface for connecting a marker, a marker may then be connected to the stud, or may be connected at a later stage in the process.

Next, at block 505, the plurality of studs may be connected together with adjustable joints. For example, each stud can be mechanically connected to the next or adjacent stud by means of a joint (such as a three-axis joint with length control) such that the studs and their joints together form an adjustable “chain.” At block 507, the surgeon can then adjust the joints and the studs attached thereto in order to place the vertebrae in a desired alignment. Once the vertebrae are in the desired alignment, at block 509, the positions of the various studs can be registered in an augmented reality system, such as by using the head-mounted device 28, 70. For example, the head-mounted device 28, 70 may be used to detect the positions of the markers, reflective spheres, and/or the like coupled to the studs, and then use the known geometry of the studs to derive positions of the pedicle screws 308. Detection of the positions can be accomplished using any of the techniques discussed above, and/or any of the techniques disclosed in the patents and publications incorporated by reference herein. This includes, but is not limited to structured light, X-ray at two positions, stereoscopic imaging, and other optical and/or IR image processing techniques.

At block 511, now that the positions of the studs, and thus the pedicle screws 308, are known, the system can be configured to analyze these positions to determine a desired bent rod shape that will result in a rod 310 that can pass through each of the pedicle screws 308 and result in aligning the spine 302 into the desired alignment. For example, the system may generate a set of points that define a curved line that defines, for example, a centerline along which the bent rod should pass.

Finally, at block 513, a bent rod can be generated using the desired rod shape output from block 511. In some embodiments, the bent rod can be generated automatically, such as by outputting the desired rod shape from block 511 to an automated bending device that creates the desired bent rod shape. Once the bent rod is generated, the surgeon can then proceed with implanting the rod, such as by using any of the processes discussed above, such as the processes described with reference to FIG. 4A, 4B, or 4C.

Similar to as discussed above with reference to the processes of FIGS. 4A-4C, conducting the process of FIG. 5 during the surgical procedure and/or in real time, as opposed to determining the rod shape in preoperative planning, can have a number of benefits, such as increased efficiency, increased accuracy, reduced preoperative planning requirements, reduced overall time required to conduct a surgical intervention, and/or the like.

### Example AR Overlay Views

Turning now to FIGS. 6A-6D, these figures illustrate schematically an example sequence of augmented reality overlays that may be displayed on, for example, portion 33 of the see-through displays discussed above and shown in FIG. 2A, during insertion of a rod 310 through pedicle screws 308. Specifically, in this sequence of four overlays, the upper rod 310 has already been placed, and the lower rod 310 is in the process of being placed. FIG. 6A shows the lower rod 310 having been passed through a first pedicle screw 308, FIG. 6B shows the lower rod 310 having been passed through a second pedicle screw 308, FIG. 6C shows the lower rod 310 having been passed through a third pedicle screw 308, and FIG. 6D shows the lower rod 310 in its final position, having passed through the final pedicle screw 308.

Each of the augmented reality overlay images depicts the spine 302, pedicle screws 308, rods 310 (including the distal tip 311 of the rod and a body 313 of the rod 310), and one or more navigational guides (e.g., guidance attributes) 620, 622. It should be noted that, in these schematic views, a representation of the entire visible portion of the pedicle screws 308 and rods 310 are depicted. However, some embodiments may merely show, for example, an indication of where the head of a pedicle screw 308 is as an indication of where the distal tip 311 of the rod 310 is, without showing the rod 310 itself (and/or without showing portions of the body 313 or the rod 310), and/or the like. For example, as discussed above, the process flow depicted in FIG. 4A would illustrate only the distal tip 311 of the rod 310, whereas the process flows depicted in FIGS. 4B and 4C may illustrate both the distal tip 311 and at least a portion of the body 313 of the rod 310.

Navigational guides 622, visible in FIGS. 6A, 6B, and 6C, comprise a directional indicator, in this case an arrow. For example, the arrow may be used to indicate to the surgeon the general direction the distal tip 311 should travel in order to properly align with and pass through the next pedicle screw 308. The orientation of the arrow may be, or may be based on, a guidance attribute determined in block 413 of processes 400, 402, 404, and/or the like, discussed above. Further, navigational guides 620 are visible in FIGS. 6A, 6B, and 6C. Navigational guides 620 comprise a distance indicator, in this case indicating a distance, in millimeters, between the distal tip 311 and the next pedicle screw 308. The distance shown in navigation guides 620 may be, or may be based on, a guidance attribute determined in block 413 of processes 400, 402, 404, and/or the like, discussed above. It should be noted that the specific navigational guides 620, 622 depicted in FIGS. 6A-6C are not intended to be limiting, and various embodiments may include other types of navigational guides, may position the navigational guides differently, may display the information depicted by these navigational guides in a different manner, and/or the like. Further, as discussed above, the guidance attributes and/or navigational guides may desirably be determined in real time based on direct or indirect tracking of the implantable device (e.g., the distal tip 311 and/or other parts of rod 310) and of one or more anchors (e.g., pedicle screw 308), without using or requiring a preoperative plan (such as a planned final installed position of the implantable device and/or a planned installation path or trajectory of the implantable device determined prior to the surgical procedure). That said, some embodiments may at least partially use such preoperative planning.

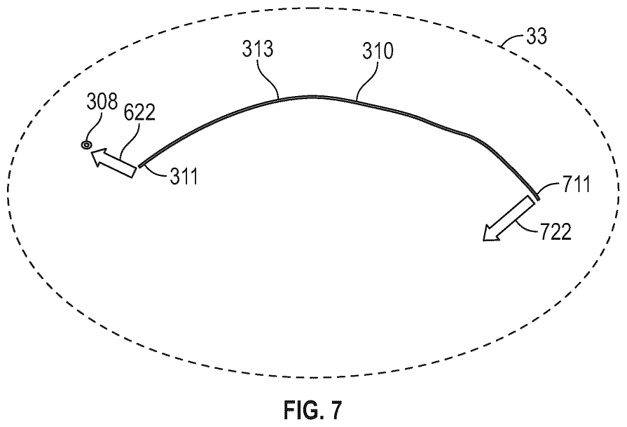

Turning now to FIG. 7, this figure illustrates schematically another version of an augmented reality overlay that may be displayed on, for example, portion 33 of the see-through displays discussed above and shown in FIG. 2A, during insertion of a rod 310 through a pedicle screw 308. This diagram illustrates many of the same or similar features as the overlays of FIGS. 6A-6D, discussed above, and the same or similar reference numbers are used to refer to the same or similar elements. One difference in the AR overlay of FIG. 7 is that a directional indicator 722 is depicted at the proximal end 711 of the rod 310, in addition to the navigational guide (directional indicator in this example) 622 at the distal end 311 of the rod 310. The additional directional indicator 722 at the proximal end of the rod can be beneficial, for example, since that is the portion of the rod 310 that will be more directly interacted with by the surgeon in order to cause movement of the distal end 311. Further, because the system may know the overall shape of the body 313 of the rod 310 (e.g., using any of the techniques discussed above for determining the shape of the body 313 of the rod 310), the system can determine what type or direction of movement of the proximal end 711 (e.g., the type or direction of movement indicated by directional indicator 722) will result in a desired type or direction of movement of the distal end 311 (e.g., the type or direction of movement indicated by directional indicator 622).

Any of the overlays shown in FIGS. 6A-6D, discussed above, may be adapted to include a proximal end directional indicator 722, such as is shown in FIG. 7. Further, some embodiments may display a directional indicator 722 at the proximal end 711 of the rod 310, without the directional indicator 622 at the distal end 311.

In addition to arrows and distances, as discussed above, various embodiments may also or alternatively include other types of navigation guides and/or directional indicators. For example, some embodiments may include an angle in addition to or in lieu of a distance. Further, in some embodiments, the guidance indicators may be shown in a 2D slice multiplanar reconstruction (MPR) view.

### Additional Depth Sensing Information

In some embodiments, the systems and methods described herein that include depth sensing capabilities may be used to measure the distance between professional 26 and a tracked element of the scene, such as bone marker 60, marker 38 and/or tool marker 40. For example, a distance sensor comprising a depth sensor configured to illuminate the ROI with a pattern of structured light (e.g., via a structured light projector) can capture and process or analyze an image of the pattern on the ROI in order to measure the distance. The distance sensor may comprise a monochromatic pattern projector such as of a visible light color and one or more visible light cameras. Other distance or depth sensing arrangements described herein may also be used. In some embodiments, the measured distance may be used in dynamically determining focus, performing stereo rectification and/or stereoscopic display. These depth sensing systems and methods may be specifically used, for example, to generate a digital loupe for an HMD such as DMD 28 or 70.

According to some embodiments, the systems and methods described herein that include depth sensing or depth mapping capabilities may be used to monitor change in depth of soft tissue relative to a fixed point to calculate the effect and/or pattern of respiration or movement due to causes other than respiration. In particular, such respiration monitoring may be utilized to improve the registration with the patient anatomy and may make it unnecessary to hold or restrict the patient's breathing. When operating on a patient during surgery, patient breathing causes movement of the soft tissues, which in turn can cause movement of some of the bones. For example, when an anchoring device such as a clamp is rigidly fixed to a bone, this bone does not move relative to the clamp, but other bones may. A depth sensor or using depth sensing as described herein to measure the depth of one or more pixels (e.g., every pixel) in an image may allow identifying a reference point (e.g., the clamp or a point on the bone the clamp is attached to) and monitoring of the changing depth of any point relative to the reference point. The change in depth of soft tissue close to a bone may be correlated with movement of the bone using this information, and then this offset may be used, inter alia, as a correction of the registration or to warn of possible large movement. Visual and/or audible warnings or alerts may be generated and/or displayed. Alternatively, or additionally, the depth sensing systems and methods described herein may be used to directly track change in depth of bones and not via soft-tissue changes.

According to some embodiments, identifying a change in depth of soft tissue at the tip of a surgical or medical instrument via the depth sensing described herein may be used as a measure of the amount of force applied. Depth sensing may be used in place of a haptic sensor and may provide feedback to a surgeon or other medical professionals (e.g., for remote procedures or robotic use in particular). For example, in robotic surgery, the amount of pressure applied by the robot may be a very important factor to control and replaces the surgeon's feel (haptic feedback). To provide haptic feedback, a large force sensor at the tip of the instrument may be required. According to some embodiments, the instrument tip may be tracked (e.g., navigated or tracked using computer vision) and depth sensing techniques may be used to determine the depth of one or more pixels (e.g., every pixel) to monitor the change in depth of the soft tissue at the instrument tip, thus avoiding the need for a large, dedicated force sensor for haptic, or pressure, sensing. Very large quick changes may either be the instrument moving towards the tissue or cutting into it; however, small changes may be correlated to the pressure being applied. Such monitoring may be used to generate a function that correlates change in depth at the instrument tip to force and use this information for haptic feedback.

### Additional Information

In some embodiments, the system comprises one or more of the following: means for depth sensing or depth mapping (e.g., a structured light projector and a camera, multiple cameras, a time-sensitive detector or detector array), means for generating a 3D model (e.g., a calibrated tracing instrument, a depth sensor, a tomographic imaging device, a fluoroscopy device), means for tracking (e.g., one or more cameras, a light source and a sensor, one or more infrared tracking systems, one or more markers, a depth sensor, an inertial measurement unit), etc.

### Conclusion and Terminology

Several embodiments of the invention, described herein, are particularly advantageous because they include one, several, or all of the following benefits: (i) increasing accuracy of implantation of medical devices, (ii) reducing the time taken to do so, (iii) reducing risk to the patient, and/or (iv) enabling completion of more complicated procedures that may otherwise be difficult or impossible to do in a minimally invasive fashion.

Although the drawings and embodiments described above relate specifically to surgery on the spine, the principles of the present disclosure may similarly be applied in other sorts of surgical procedures or non-surgical medical treatment and/or diagnostic procedures, such as operations performed on the cranium and various joints (e.g., shoulders, knees, ankles, elbows, sacroiliac joints) or other bones, as well as dental procedures or ear-nose-throat procedures. It will thus be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid-state memory, optical discs, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage, such as, for example, volatile or non-volatile storage.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

As used herein, “generate” or “generating” may include specific algorithms for creating information based on or using other input information. Generating may include retrieving the input information, such as from memory or as provided input parameters to the hardware performing the generating. Once obtained, the generating may include combining the input information. The combination may be performed through specific circuitry configured to provide an output indicating the result of the generating. The combination may be dynamically performed, such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network location, file system location, or the like.

Any process descriptions, elements, or blocks in the flow diagrams described herein and/or depicted in the attached figures should be understood as potentially representing modules, segments, or portions of code that include one or more executable instructions for implementing specific logical functions or steps in the process. Alternate implementations are included within the scope of the embodiments described herein, in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general-purpose computers. For example, the methods described herein may be performed by the processors 32, 45 described herein and/or any other suitable computing device. The methods may be executed on the computing devices in response to execution of software instructions or other executable code read from a tangible computer readable medium. A tangible computer-readable medium is a data storage device that can store data that is readable by a computer system. Examples of computer-readable mediums include read-only memory, random-access memory, other volatile or non-volatile memory devices, CD-ROMs, magnetic tape, flash drives, and optical data storage devices.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As it is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

What is claimed is:

1. An augmented reality surgical display system for guiding implantation of a medical device, the system comprising:
a see-through display configured to overlay augmented reality images onto reality;
one or more cameras; and
at least one processor configured to:
detect and store in a memory a plurality of medical device fixation locations in a patient;
calibrate a medical device distal tip location with respect to a handle marker;
track, using the one or more cameras during a surgical procedure, a location of the handle marker;
determine a location of the medical device distal tip based on the tracked location of the handle marker;
generate one or more guidance attributes based on the determined location of the medical device distal tip and the stored locations of one or more of the plurality of medical device fixation locations; and
display on the see-through display, aligned with reality, the following:
an indication of the determined location of the medical device distal tip;
an indication of locations of one or more of the plurality of medical device fixation locations; and
the one or more guidance attributes.

2. The system of claim 1, wherein the at least one processor is further configured to:
generate a 3D virtual model of a body of the medical device; and
further display on the see-through display, aligned with reality, at least a portion of the generated 3D virtual model of the body of the medical device.

3. The system of claim 2, further comprising:
a depth sensor,
wherein the at least one processor is configured to generate the 3D virtual model of the body of the medical device using data output from the depth sensor.

4. The system of claim 2, wherein the at least one processor is configured to generate the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images.

5. The system of claim 1, wherein the generation of the one or more guidance attributes comprises comparing the determined location of the medical device distal tip to a location of one of the plurality of medical device fixation locations to determine a direction in which the medical device distal tip should be moved to reach the one of the plurality of medical device fixation locations.

6. The system of claim 1, wherein the generation of the one or more guidance attributes comprises comparing the determined location of the medical device distal tip to a location of one of the plurality of medical device fixation locations to determine a distance by which the medical device distal tip should be moved to reach the one of the plurality of medical device fixation locations.

7. The system of claim 1, wherein:
each of the plurality of medical device fixation locations comprises a fixation location of a pedicle screw, and
the medical device comprises a rod shaped to be affixed to the plurality of medical device fixation locations.

8. The system of claim 1, wherein the one or more guidance attributes comprises one or more directional indicators indicative of one or more of the following: a direction in which the medical device distal tip should be moved or a direction in which a proximal end of the medical device should be moved.

9. The system of claim 1, wherein the one or more guidance attributes comprise a distance of the medical device distal tip to one of the plurality of medical device fixation locations.

10. An augmented reality surgical display system for guiding implantation of a medical device, the system comprising:
a see-through display configured to overlay augmented reality images onto reality;
one or more cameras;
a depth sensor; and
at least one processor configured to:
detect and store in a memory a plurality of medical device fixation locations in a patient;
track, using the depth sensor during a surgical procedure, a location of a distal tip of the medical device;
generate one or more guidance attributes based on the tracked location of the distal tip of the medical device and the stored locations of one or more of the plurality of medical device fixation locations; and
display on the see-through display, aligned with reality, the following:
an indication of the tracked location of the distal tip of the medical device;
an indication of locations of one or more of the plurality of medical device fixation locations; and
the one or more guidance attributes.

11. The system of claim 10, wherein the at least one processor is further configured to:
generate a 3D virtual model of a body of the medical device; and
further display on the see-through display, aligned with reality, at least a portion of the generated 3D virtual model of the body of the medical device.

12. The system of claim 11, wherein the at least one processor is configured to generate the 3D virtual model of the body of the medical device using data output from the depth sensor.

13. The system of claim 11, wherein the at least one processor is configured to generate the 3D virtual model of the body of the medical device using data from at least one of the following processes: tracing of the body with a calibrated instrument, detection of a shape of the body with fluoroscopy imaging, detection of a shape of the body with CT imaging, or detection of a shape of the body with two or more X-ray images.

14. The system of claim 10, wherein the generation of the one or more guidance attributes comprises comparing the tracked location of the distal tip of the medical device to a location of one of the plurality of medical device fixation locations to determine a direction in which the distal tip of the medical device should be moved to reach the one of the plurality of medical device fixation locations.

15. The system of claim 10, wherein the generation of the one or more guidance attributes comprises comparing the tracked location of the distal tip of the medical device to a location of one of the plurality of medical device fixation locations to determine a distance by which the distal tip of the medical device should be moved to reach the one of the plurality of medical device fixation locations.

16. The system of claim 10, wherein:
each of the plurality of medical device fixation locations comprises a fixation location of a pedicle screw, and
the medical device comprises a rod shaped to be affixed to the plurality of medical device fixation locations.

17. The system of claim 10, wherein the one or more guidance attributes comprise one or more directional indicators indicative of one or more of the following:
a direction in which the distal tip of the medical device should be moved, or
a direction in which a proximal end of the medical device should be moved.

18. The system of claim 10, wherein the one or more guidance attributes comprise a distance of the distal tip of the medical device to one of the plurality of medical device fixation locations.

19. The system of claim 10, wherein the at least one processor is configured to detect the locations of the plurality of medical device fixation locations using data output from the depth sensor.

20. A method of guiding implantation of a medical device, the method comprising:
detecting and storing, in a memory, locations of a plurality of medical device fixation locations in a patient;
detecting a relationship between a medical device distal tip and a handle marker, to calibrate the medical device distal tip with respect to the handle marker;
tracking, using one or more cameras during a surgical procedure, a location of the handle marker;
determining a location of the medical device distal tip based on the tracked location of the handle marker;
generating, by at least one processor, one or more guidance attributes based on the determined location of the medical device distal tip and the stored locations of one or more of the plurality of medical device fixation locations; and
displaying on a see-through display, aligned with reality, the following:
an indication of the determined location of the medical device distal tip;
an indication of locations of one or more of the plurality of medical device fixation locations; and
the one or more guidance attributes.
